(19) United States
(12) Patent Application Publication
(10) Pub. No.: US 2023/0241385 A1
(43) Pub. Date: Aug. 3, 2023

(54) Transcranial Stimulation to Treat DMN Dysfunction in Normal and Abnormal Aging

(71) Applicant: THE FLORIDA STATE UNIVERSITY RESEARCH FOUNDATION, INC., Tallahassee, FL (US)

(72) Inventors: Wen Li, Tallahassee, FL (US); Kevin Clancy, Tallahassee, FL (US)

(21) Appl. No.: 18/103,190

(22) Filed: Jan. 30, 2023

Related U.S. Application Data
(60) Provisional application No. 63/305,158, filed on Jan. 31, 2022.

(51) Int. Cl. A61N 1/36; A61N 1/04

(52) U.S. Cl. CPC A61N 1/36025; A61N 1/36034; A61N 1/0456

(57) ABSTRACT

Methods are provided for treating disorders associated with default mode network (DMN) dysfunction in a patient, where the methods include administering alpha-frequency transcranial alternating current stimulation (α-tACS) to the brain of the patient via at least one electrode, and where the α-tACS is effective to regulate DMN dysfunction and treat disorders associated therewith. Diseases and disorders associated with DMN dysfunction include normal or abnormal aging, cognitive impairment, or neuropsychiatric disorders such as Alzheimer's disease, schizophrenia, autism, or posttraumatic stress disorder (PTSD).

### CROSS-REFERENCE TO RELATED APPLICATIONS

This application claims priority to U.S. Provisional Patent Application No. 63/305,158, filed on Jan. 31, 2022, which is incorporated herein by reference.

### STATEMENT REGARDING FEDERALLY SPONSORED RESEARCH OR DEVELOPMENT

This invention was made with government support under grant number R01MH093413 awarded by the National Institutes of Health. The government has certain rights in the invention.

### BACKGROUND

It is widely recognized that the brain self-organizes into large-scale intrinsic networks. Such intrinsic organization is so fundamental to normal neural functioning that it commands 60 to 80% of the brain's energy. Two main mechanisms—intrinsic interregional connectivity and interneuronal synchrony—are thought to underpin the brain's organization. The default mode network (DMN), emerging from intrinsic interregional connectivity crisscrossing a large extent of the brain, occupies the apex of intrinsic connectivity networks and dominates the brain's intrinsic activity.

Accordingly, the DMN supports advanced human mental faculties (e.g., consciousness, self-reference, social inference, remembering the past, and expecting the future), while its dysregulation leads to major neuropsychiatric disorders (e.g., DMN hyperconnectivity in major depression and hypoconnectivity in Alzheimer's disease, schizophrenia, and posttraumatic stress disorder). Currently, there is no effective intervention for DMN dysfunction. Mechanisms regulating the DMN remain elusive, while effective interventions for DMN dysregulation are lacking. Accordingly, an effective intervention for DMN dysfunction is needed.

### BRIEF SUMMARY

In one aspect, methods are provided for treating disorders associated with default mode network (DMN) dysfunction in a patient, where the methods include administering alpha-frequency transcranial alternating current stimulation (α-tACS) to the brain of the patient via at least one electrode, and where the α-tACS is effective to regulate DMN dysfunction and treat disorders associated therewith. In some particular embodiments, the α-tACS is administered to the occipitoparietal cortex of the brain of the patient. In some other particular embodiments, the at least one electrode is a plurality of electrodes positioned on and/or around the midline of the occipitoparietal cortex.

In some preferred embodiments, the plurality of electrodes includes a central electrode positioned on the midline of the occipitoparietal cortex, at least two right electrodes positioned right of the midline of the occipitoparietal cortex, and at least two left electrodes positioned left of the midline of the occipitoparietal cortex. In some other preferred embodiments, the α-tACS is a 2 mA sinusoidal current oscillating a 10 Hz, administered for at least 10 minutes, and preferably 20 minutes.

In another aspect, methods are provided for regulating default mode network (DMN) functioning in the brain, where the methods include forming a closed-loop circuit comprising two or more electrode and applying alpha-frequency transcranial alternating current stimulation (α-tACS) to the brain via the closed-loop circuit of electrodes, where the α-tACS targets a primary rhythm of neural synchrony in the brain associated with DMN functioning. In some particular embodiments, the closed-loop circuit of electrodes includes a central electrode and four peripheral electrodes. In some other particular embodiments, the primary rhythm of neural synchrony targeted by the α-tACS is the alpha-frequency, preferably 8 to 12 Hz oscillations.

In yet another aspect, systems for delivery alpha-frequency transcranial alternating current stimulation (α-tACS) to the brain are provided, including at least one electrode configured to be removably attachable to the patient and an apparatus configured to generate α-tACS, where the apparatus is operationally connected to the at least one electrode, such that the α-tACS is delivered to the patient via the at least one electrode. In some particular embodiments, the at least one electrode is a plurality of electrodes configured to target the occipitoparietal cortex, and includes a central electrode configured to target the midline of the occipitoparietal cortex, at least one right electrode configured to target a portion of the occipitoparietal cortex to the right of the midline, and at least one left electrode configured to target a portion of the occipitoparietal cortex to the left of the midline.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components are not necessarily drawn to scale.

FIG. 1 is a schematic diagram of an exemplary electrode array and method for a-tACS stimulation, as disclosed herein.

FIG. 2 is a depiction of a standard versus realistic head model.

FIG. 3A depicts the localization of the source of alpha power in the bilateral occipitoparietal cortex (display threshold P<0.001 familywise error rate, k=20; Top) and the localization of the source-level alpha power increase after tACS to the right occipitoparietal cortex (display threshold P<0.05, k=10; bottom).

FIG. 3B depicts topography of alpha (8 to 12 Hz) power before and after stimulation.

FIG. 3C depicts spectral waveforms averaged across the right occipitoparietal electrodes, demonstrating specific increases in the Active (versus Sham) groups, which were restricted to the alpha frequency (light gray box); the dark gray bar indicates frequency bins (0.25 Hz each) showing significant tACS effects; Ribbon=SEM.

FIG. 3D shows violin plots for alpha power over the left and right electrodes, indicating significant right posterior power increase in the Active group.

FIG. 3E depicts the right-hemisphere P→F GC waveforms, demonstrating specific increases in the Active (versus Sham) group, which were restricted to the alpha frequency (light gray box); the dark gray bar indicates frequency bins (0.5 Hz each) showing significant tACS effects; Inset shows ipsilateral electrode pairs used for alpha GC; Ribbon=SEM.

FIG. 3F shows violin plots for alpha-frequency P→F GC in the left and right hemispheres, indicating significant increase in the Active group.

FIG. 4 is a topographical map of change (Post-Pre) in P→F a-connectivity from the right posterior sender.

FIG. 5A is a post-versus pre-stimulation matrix for the Active (lower half) and the Sham Control (upper half) groups.

FIG. 5B shows a violin plot for the Active and the Sham Control groups.

FIG. 5C depicts vPCC-seed whole-brain connectivity maps for the Active and the Sham Control groups.

FIG. 5D is a post-versus pre-stimulation matrix for the Active and the Sham Control groups.

FIG. 5E depicts whole-brain maps of tACS enhancement of a-connectivity from the PCC.

FIG. 5F is an ROI-based PCC→mPFC violin plot.

FIG. 6 is a 3D depiction of key posterior brain regions.

FIG. 7A is a scatterplot demonstrating a positive correlation between changes in BOLD vPCC-mPFC connectivity and right-hemisphere P→F alpha-frequency connectivity/GC.

FIG. 7B is a mediation model demonstrating indirect (i.e., mediation) effect of tACS group on increases in DMN connectivity through increases in right-hemisphere P→F alpha-frequency GC.

FIG. 8A is a violin plot demonstrating that tACS increased connectivity between the tACS site and mid-line DMN hubs (tACS-mPFC and tACS-vPCC).

FIG. 8B depicts tACSsite seed-based whole-brain connectivity maps, further indicating increases in tACS connectivity to mid-line DMN hubs for the Active group alone (P<0.005 uncorrected, k>10).

### DETAILED DESCRIPTION

Methods have been developed for treating certain diseases and disorders associated with DMN dysfunction in aging, and DMN dysfunction more generally. The default mode network (DMN) is the most-prominent intrinsic connectivity network in the brain, serving as a key architecture of the brain's functional organization. Conversely, dysregulated DMN is characteristic of major neuropsychiatric disorders. Interneuronal synchrony is thought to be inherently related to interregional connectivity and potentially bind and sculpt such connectivity through neural development. Importantly, the alpha (8 to 12 Hz) oscillation, the primary rhythm of intrinsic neural synchrony, has been liked to DMN functioning. In fact, rapid advances in neuroimaging and neurocomputing have brought forward mounting evidence of multifaceted physiological and functional associations between the alpha oscillation and the DMN. Physiologically, resting-state (RS) simultaneous EEG-fMRI (electroencephalography and functional MRI) recordings have revealed intrinsic positive coupling between alpha oscillations and DMN activity.

Of particular relevance, akin to its role in long-range neural communication, alpha oscillations are found to be the primary neural synchrony linking the posterior and anterior hubs of the DMN (the posterior cingulate cortex (PCC) and medial prefrontal cortex (mPFC), respectively). Functionally, alpha oscillations and the DMN are both involved in disengaging the brain from the sensory environment and maintaining the RS, while alpha desynchrony and DMN dysconnectivity, including specific disruption of alpha oscillatory PCC-mPFC connectivity, co-occur in several major neuropsychiatric disorders (e.g., Alzheimer's disease, schizophrenia, and posttraumatic stress disorder).

It was discovered that using high-definition alpha-frequency transcranial alternating current stimulation (α-tACS) to stimulate the cortical source of alpha oscillations augmented alpha oscillations and strengthened alpha connectivity within the core of the DMN. Increase in alpha oscillations mediated the DMN connectivity enhancement, thereby indicating a mechanistic link between alpha oscillations and DMN functioning. This transcranial modulation can up-regulate the DMN highlights an effective noninvasive intervention to normalize DMN functioning in various disorders.

The noninvasive systems and methods disclosed herein are particularly useful in enhancing neural functioning through the upregulation of the DMN. Accordingly, these systems and methods are useful in preventing and treating normal and abnormal aging, mild cognitive impairment, neuropsychiatric disorders (including Alzheimer's disease, schizophrenia, autism, and posttraumatic stress disorder), and other disorders associated with DMN dysfunction. Specifically, the systems and methods herein can slow down cognitive decline in normal aging, prevent the progression of Alzheimer's disease, and alleviate the effects of Alzheimer's disease, schizophrenia, or posttraumatic stress disorder, for example.

Because of the proximity of the primary source of alpha oscillations—the occipitparietal cortex—to the scalp, alpha oscillation is highly responsive to transcranial stimulation and can be a viable target for therapeutic manipulation. In particular, transcranial alternating current stimulation (tACS) applies frequency-specific sinusoidal electric currents through the scalp, which is uniquely advantageous in mimicking and entraining endogenous oscillations by tuning not only the frequency and amplitude but also the oscillatory phase. The latter, by enhancing phase synchronization, is particularly effective at facilitating interregional connectivity. Therefore, alpha oscillations may be manipulated with HD alpha-frequency tACS (α-tACS) targeting the occipitoparietal alpha source, resulting in enhanced alpha synchrony. This enhanced alpha synchrony facilitates synchronization of blood oxygen level-dependent (BOLD) fluctuations, resulting in increased DMN connectivity.

Accordingly, systems and methods for safe and non-invasive DMN regulation are provided herein. To regulate DMN functioning, α-tACS is administered to the patient through at least one electrode. In some embodiments, the at least one electrode is externally affixed to the patient's scalp. In some embodiments, electrodes are removably affixed to the scalp through an adhesive coating (e.g., a pressure sensitive adhesive known in the art) on the conductive surface of the electrode. In other embodiments, electrodes may be removably affixed to the patient using other non-invasive means, as would be understood by those skilled in the art.

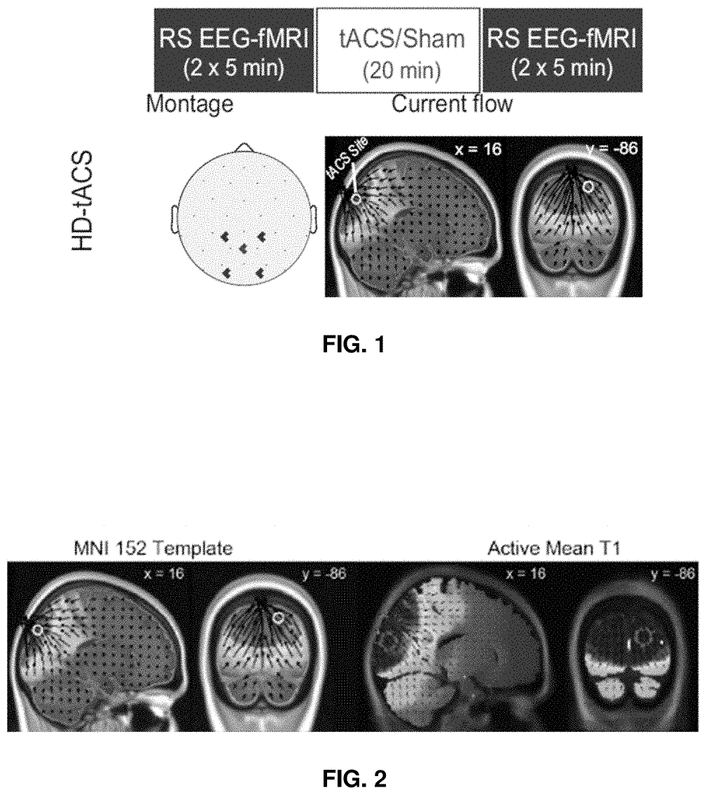

In particular, α-tACS may be administered through an array of five electrodes place near the occipitoparietal cortex (FIG. 1). In some embodiments, the array of electrodes includes a central electrode and four peripheral electrodes. The center electrode may be placed on the scalp at or near the midline of the occipitparietal cortex, while the peripheral electrodes are disposed one the scalp surrounding the central electrode. In some embodiments, two peripheral electrodes may be placed to the left of the central electrode and two peripheral electrodes may be placed to the right of the central electrode, preferably forming a square array around the central electrode. In a preferred embodiment, the electrodes form a closed-loop circuit.

The α-tACS of the alpha source in the occipitoparietal cortex not only augments alpha oscillations, but also strengthened BOLD and alpha-frequency oscillatory connectivity within the DMN. Notably, tACS-induced augmentation of posterior to frontal alpha connectivity mediates tACS enhancement of BOLD connectivity between DMN hubs, but no tACS effects emerged outside the alpha frequency of DMN. Because posterior alpha power, and posterior to frontal alpha connectivity reflect local alpha synchrony in the occipitoparietal cortex and long-range synchrony front the occipitoparietal cortex to the frontal cortex, respectively, tACS may be effective for modulation of both local and distant interareal synchrony.

By strengthening oscillatory circuits via spike timing-dependent plasticity and long-term potentiation at the synapse, α-tACS can exert immediate and lasting effects on long-range neural communication (i.e., alpha-frequency connectivity) and BOLD connectivity beyond local power enhancement via neural entrainment. The immediate effects may be effective to mitigation symptoms associated with neurodegenerative or neuropsychiatric disorders, such as Alzheimer's. For example, those undergoing α-tACS may experience slowed memory loss or increased periods of lucidity. The lasting effects may also provide long term treatment for the same, and in the context of Alzheimer's for example, may halt or significantly reduce the rate of neurological degeneration.

In some embodiments, administering α-tACS to the occipitoparietal cortex may be effective to target the primary rhythm of neural synchrony in the alpha frequency, which is generally understood to be 1 Hz to 30 Hz. In some preferred embodiments, the α-tACS targets the 8 Hz to 12 Hz oscillations in the brain. Administering α-tACS to target 8 Hz to 12 Hz oscillations may be optimal because this frequency of α-tACS does not affect alpha connectivity within the brain. This observation suggests that α-tACS primarily serves to amplify the underlying endogenous activity in the brain.

In some embodiments, the α-tACS is a 2 mA sinusoidal current oscillating at 10 Hz. In some embodiments, the 2 mA sinusoidal current is delivered equally across the electrode array (i.e., the net current from the electrodes is 2 mA, or 0.4 mA per electrode where the 5-electrode array is used). In some other embodiments, the 2 mA sinusoidal current is not delivered equally across the electrode array (i.e., the net current from the electrodes is 2 mA, but one or more electrodes in the array may be dominant sources of current). According to some alternative embodiments, the array of electrodes administers α-tACS with a net 2.5 mA sinusoidal current. The central electrode may administer 2 mA sinusoidal current, whereas the peripheral electrodes may administer a net 0.5 mA sinusoidal current.

To effectively regulate DMN function to treat or mitigate symptoms of neurodegenerative or neuropsychiatric disorders, the α-tACS may be administered over the course of minutes or hours, depending on the severity of DMN irregularity or dysfunction. In some embodiments, exposure to α-tACS for at least 10 minutes may be sufficient to improve DMN connectivity. In a preferred embodiment, the patient is exposed to α-tACS for at least 20 minutes per treatment. Treatments may be administered over a period of days, weeks, months, or years, at varying frequencies, depending a patient's particular needs. For example, patients with more severe symptoms and/or in late stages of disease progression may require more frequent treatments over an extended period of time. In contrast, patients with mild symptoms and/or in the early stages of disease progression may require fewer, less-frequent treatments.

According to some embodiments, a system for administering α-tACS is provided. In some embodiments, the system includes a stimulation apparatus (unit) and at least one electrode, preferably five electrodes. In some embodiments, the system may be designed for use by medical professionals only. In some other embodiments, the system may be designed to be suitable for home use so that patients may administer the α-tACS themselves (or with the assistance of a family member, care taker, etc.). Stimulation apparatus and electrodes are known in the art which can be adapted for delivering alpha-frequency transcranial alternating current stimulation (α-tACS) to the brain as described herein.

According to some alternative embodiments, tACS may be used to target areas of the brain other than the occipitparietal cortex. For example, tACS modulation of the posterior cingulate cortex (PCC) could result in particularly salient DMN effects. Within the midline core, there are actually two interdigitated subnetworks, the vPCC and the dorsal PCC (dPCC), with the former connected with the prefrontal cortex (PFC) hub (the anterior and ventral PFC) and the latter with the dorsal PFC. Examination of each of the ventral and dorsal subdivisions of the PCC demonstrated effects of tACS on the ventral PCC DMN connectivity, but not connectivity of the dorsal PCC. Accordingly, applying tACS to the ventral PCC, or another area associated with the core of the DMN, may be affective in upregulating alpha and/or DMN connectivity in a manner that is also effective to mitigate the symptoms of and/or treat various neurological disorders.

### EXAMPLES

The invention can be further understood with reference to the following non-limiting examples.

### Example 1. Use of α-tACS to Modulate Alpha-Frequency Activity

In total, 41 healthy volunteers (24 female, 20.8±3.2 y of age) participated in the study after providing written, informed consent. No participants reported a history of neurological or psychiatric disorders or current use of psychotropic medication. Participants were randomly assigned to two groups, an Active (n=21) and a Sham group (n=20). Two participants (Active n=2) terminated their participation prematurely due to discomfort in the scanner. Three participants (Active n=2, Sham n=1) were excluded from fMRI analyses due to excessive motion (defined by >5% of scans exceeding a framewise displacement index of 0.5 mm), resulting in a final sample of 36 participants for fMRI analyses (Active n=17, Sham n=19). Four separate participants (Active n=1, Sham n=3) were excluded from EEG analyses due to significant artifacts, resulting in a final sample of 35 participants for EEG analyses (Active n=18, Sham n=17). Participants in the two groups did not differ in age or gender distribution (p's>0.50).

The experiment consisted of three phases: pre-stimulation RS recordings, tACS/Sham stimulation, and post-stimulation RS recordings. In both pre- and post-stimulation phases, participants underwent two successive 5-min simultaneous EEG-fMRI scans (with eyes open and fixated on a central crosshair). The MR-compatible stimulation was fully integrated with simultaneous EEG-fMRI recordings such that it did not require transition between the RS recording phases.

Alpha-frequency stimulation was administered with a ±2 mA sinusoidal current oscillating at 10 Hz using an MR-compatible HD tACS system (Soterix Medical, New York, N.Y.). Stimulation electrodes were placed in a 4×1 montage over midline occipitoparietal sites, with 4 surrounding+1 central electrodes forming a closed circuit (FIG. 1A). These electrode sights were selected to maximally target the primary cortical source of alpha oscillations-occipitoparietal cortex.

α-tACS or Sham stimulation was administered for 20 min. To minimize awareness of experimental condition, the Sham group received α-tACS for 10 s at the beginning and the end of the phase. All participants completed a standard continuous performance task, which, by maintaining alertness, would enhance α-tACS efficacy. All participants were first told they would receive electrical stimulation and were informed of their true assignment during the debriefing at the end of experiment. Participants' blindness to the group assignment was confirmed via a funnel interview at the debriefing, which was further corroborated with the Adverse Effects Questionnaire at the end of experiment. Specifically, the Active and Sham groups showed no difference in their subjective experiences during the stimulation period (t=0.81, P=0.423) or the degree to which they attributed these sensations to the stimulation (t=1.23, P=0.225).

Use of EEG and fMRI to Assess Impact of α-tACS on Alpha-Frequency

### EEG Acquisition and Analyses

EEG data were recorded simultaneously with fMRI using a 64-channel MR-compatible EEG system (Brain Products GmbH, Germany). An additional electrode was placed on the participant's upper back to record electrocardiogram for cardioballistic artifact correction. The EEG recording system was synchronized with the fMRI scanner's internal clock throughout acquisition to facilitate successful removal of MR gradient artifact.

Cardioballistic and gradient artifact corrections were performed offline using an average artifact template subtraction method as implemented in Brain Vision Analyzer 2.0 (Brain Products GmbH). The gradient artifact template was constructed with a sliding-window approach over 41 consecutive volumes. For cardioballistic artifact correction, ECG R peaks were first identified using a semi-automatic detection approach (i.e., automated peak detection combined with visual inspection) and then used to construct a delayed average artifact template over 41 consecutive heartbeat events, which was then subtracted from the EEG data. Artifact-corrected EEG data were then band-pass filtered between 0.5 and 50 Hz and down-sampled to 250 Hz before submission to the Fully Automated Statistical Thresholding for EEG artifact Rejection (FASTER) algorithm for further artifact correction. The FASTER algorithm (implemented in EEGLAB) corrected EEG data for remaining physiological and non-physiological artifacts in each channel, epoch, and independent component. Output data were epoched into segments of 1.8 s (i.e., TR length), centered on the onset of each fMRI scan.

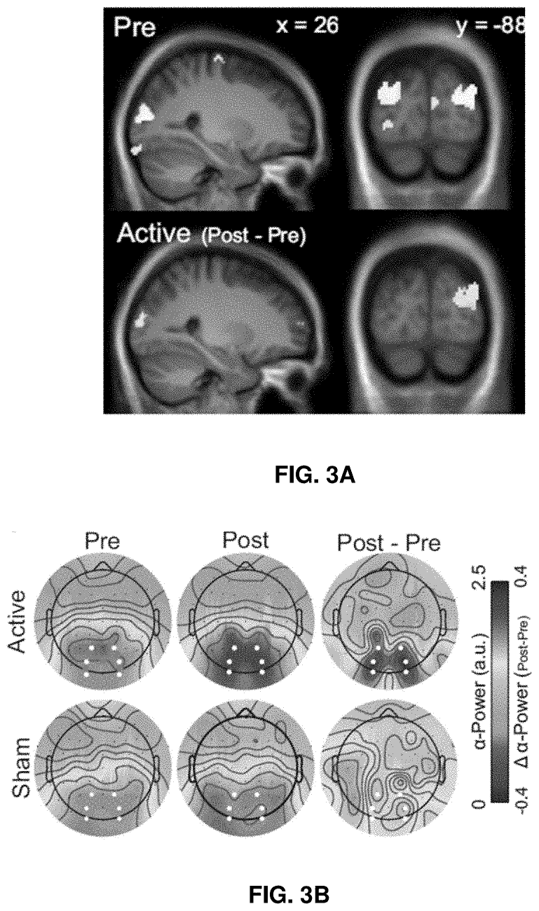

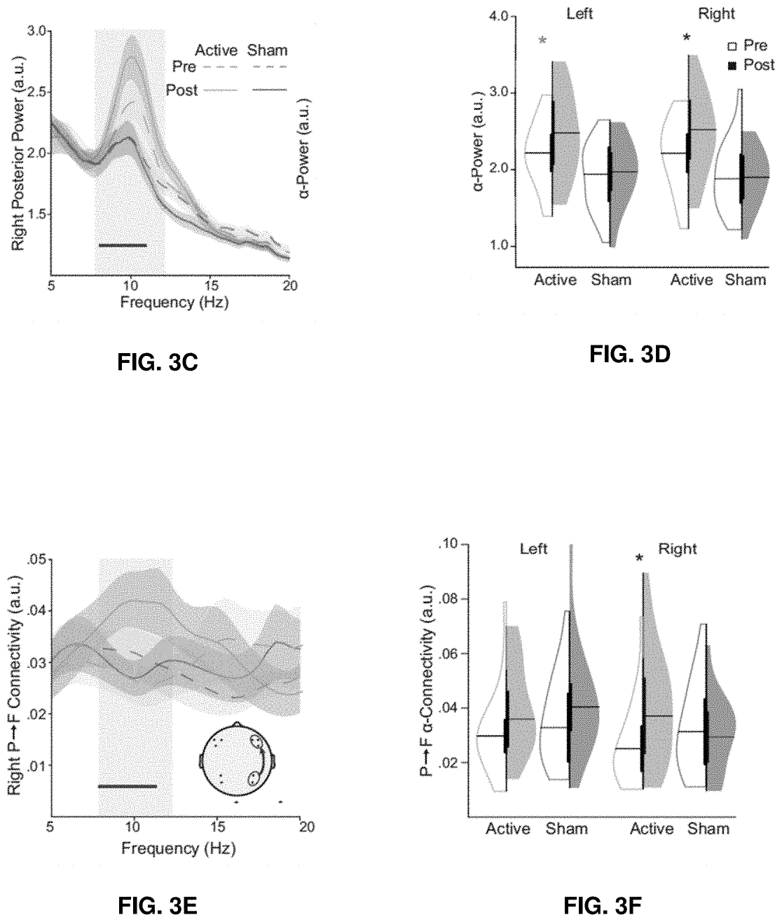

Power of alpha-frequency oscillations were computed using the multitaper spectral estimation technique. Alpha power was normalized by the mean power of the entire frequency spectrum (1-50 Hz) within each epoch, and then averaged across occipitoparietal electrodes where alpha is maximally distributed (FIG. 3B).

Directed alpha-frequency connectivity was assessed using GC analysis. EEG data were transformed to reference-free current source density data (CSD) using the surface Laplacian algorithm. CSD data from ipsilateral posterior-frontal pairs were then submitted to bivariate autoregressive (AR) modeling, from which Granger causality spectra were derived and averaged across the alpha frequency (8-12 Hz) for each posterior-frontal pairs. A model order of 20 (80 msec in time for a sampling rate of 250 Hz) was chosen in a two-step process: 1) Akaike Information Criteria (AIC) and 2) comparing spectral estimates obtained from the Fourier-based AR model on data pooled across all subjects. Given a priori hypotheses on the posterior→frontal dominance of alpha-frequency connectivity at rest and the posterior target of the α-tACS, analyses were constrained to the posterior→frontal direction.

Source-level analysis of alpha activity was performed using the Fieldtrip toolbox implemented in the Statistical Parametric Mapping Software, 12th Edition (SPM12), with the head model defined by each participant's T1 scan. The multiple sparse priors algorithm was used to generate the inverse solution. Induced alpha power was extracted using a series of Morlet wavelet projectors within each individual epoch and then averaged across epochs, as implemented by default in SPM12.

To maintain the temporal resolution needed to compute GC, source-based alpha-frequency connectivity was assessed based on ROI timeseries derived from Exact Low-Resolution Electromagnetic Tomography (eLORETA). These ROI time-series were then submitted to Granger causality analysis using the same criteria described above. Granger causality values were then natural log transformed before submission to statistical analyses. ROIs consisted of 10-mm spheres around bilateral cortical DMN hubs—posterior cingulate cortex (PCC), medial prefrontal cortex (mPFC), and angular gyrus (ANG)—and the maximal tACS stimulation site (i.e., “tACS site”). Given the low spatial resolution of source-level EEG analysis, ventral and dorsal PCC were combined into a single ROI of PCC.

### MRI Acquisition and Preprocessing

Gradient-echo T2-weighted echoplanar images were acquired on a 3T Siemens Prisma MRI scanner using a 64-channel head coil with axial acquisition. Imaging parameters included TR/TE: 1,800/22.40 ms; slice thickness 1.8 mm; gap 0.45 mm; in-plane resolution/voxel size 1.8×1.8 mm; multiband acceleration factor=2; GeneRalized Autocalibrating Partial Parallel Acquisition (GRAPPA) acceleration factor=2. A high-resolution (0.9×0.9×0 9 mm3) three-dimensional Magnetization Prepared Rapid Acquisition Gradient Echo (3D-MPRAGE) T1 scan was also acquired. Imaging data were preprocessed using SPM12, including slice-time correction, spatial realignment, and normalization using Diffeomorphic Anatomical Registration Through Exponentiated Lie algebra.

To further remove artifacts potentially contributing to spurious RS activity variance, additional preprocessing was implemented using the Data Processing Assistant for Resting-State fMRI (DPARSFA) toolbox: 1) mean centering and whitening of timeseries; 2) temporal bandpass (0.01 to 0.08 Hz) filtering; 3) general linear modeling to partial out head motion with 24 nuisance variables (six head motion parameters each from the current and previous scan and their squared values); and 4) scrubbing of significant motion (“spikes”) based on framewise displacement index (FDi>0.5 mm). In the absence of a well-established index (equivalent to EEG power) of resting-state fMRI activity, the fMRI analysis focused on connectivity, a fundamental metric for functioning of a large-scale network.

### Regions of Interest (ROIs)

ROIs consisted of key nodes of the DMN, including the midline hubs, PCC and mPFC, and the bilateral ANG. Masks for the mPFC and ANG ROIs were drawn from the Willard Atlas. The PCC hub consists of functionally dissociable ventral and dorsal subdivisions, specifically the vPCC primarily linking DMN nodes and the dPCC with extensive extra-DMN connections. Masks for the vPCC and dPCC subdivisions were extracted individually from the Brainnetome Atlas. Since these masks were not applicable for low-resolution source EEG tomography (as in -eLORETA), 10-mm spheres were applied around their respective coordinates established in the literature. The tACSsite was also included as an ROI, represented by a 10-mm sphere centered on the voxel with the maximal electric field (x, y, z=16, −86, 36) (FIG. 1).

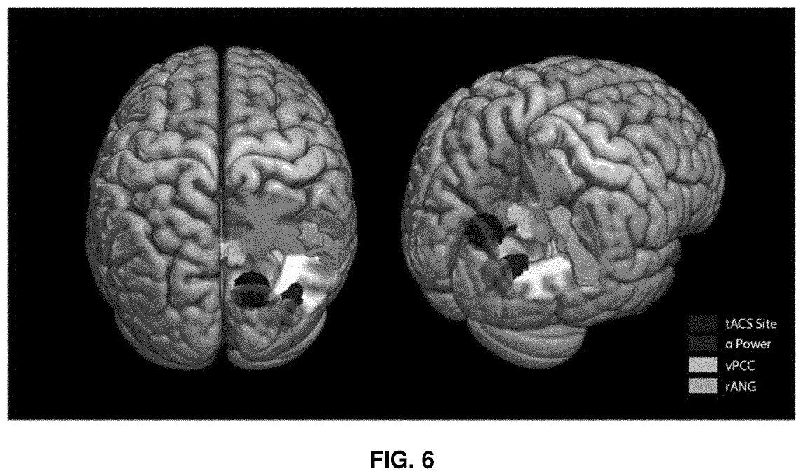

The ROI analysis encompassed three sets of data (scalp EEG, source EEG, and fMRI). Scalp EEG was directly measured and thus used to validate the α-tACS manipulation, i.e., directly indexing augmentation of alpha power and connectivity (canonical indices of alpha oscillations). Source EEG was estimated based on scalp EEG to reflect intracranial alpha oscillations. Specifically, source EEG and fMRI in the DMN were examined to test the main hypothesis-tACS upregulation of DMN connectivity. To explore mechanisms of tACS, fMRI connectivity was further analyzed with the tACS site. The distinct nature of these datasets and their respective tests warranted three sets of ROIs (as summarized in Table 1). FIG. 6 further illustrates the spatial relation of these a priori ROIs in the posterior brain and the peak cluster of alpha power increase.

TABLE 1
a priori ROIs for scalp/source EEG and fMRI corresponding tests

| Tests | Data | ROIs |
|---|---|---|
| Validation of α-tACS | Scalp EEG-α-power | Left/right |
| | | occipitoparietal |
| | | electrodes (FIG. 1C) |
| | Scalp EEG-α- | Ipsilateral (left/right |
| | connectivity | hemisphere) posterior- |
| | | frontal pairs (approx. |
| | | PO3/PO4 & F5/F6; |
| | | FIG. 1F) |
| Hypothesis testing | fMRI | d/vPCC (Brainnetome |
| (tACS upregulation | | atlas) |
| of DMN) | | mPFC (Willard atlas) |
| | | I/rANG (Willard atlas) |
| | Source EEG | PCC (0, −50, 30; |
| | | 10-mm sphere) |
| | | mPFC (0, 50, 0; 10-mm |
| | | sphere) I/rANG (−50/50, |
| | | 50, 25; 10-mm sphere) |
| Exploratory: (enhanced | fMRI | tACS site (modelled |
| tACSₛᵢₜₑ connectivity) | | maximal electric field) |
| | | (16, −86, 36; 10-mm |
| | | sphere) |

Note:
Scalp alpha power and GC ROIs were based on a priori electrodes; DMN ROIs were based on Brainnetome and Willard atlases for fMRI and a priori coordinates for source EEG. tACS site ROI was based on the maximal electric field estimated according to the current tACS montage.

Analysis of EEG and fMRI Data to Determine Impact of α-tACS on Alpha-Frequency Activity

Pre- and post-stimulation fMRI timeseries from each of the six ROIs were submitted to Pearson's correlation analysis to construct a 5×5 correlation matrix for each session for each participant. The pairwise correlation coefficients were Fisher Z transformed before submission to statistical analyses. Seed-based whole-brain connectivity, with PCC and tACS site as seeds, was further evaluated to ascertain the extent of connectivity changes.

The efficacy of tACS was first established by examining changes in alpha power and P→F alpha connectivity. ROI-based connectivity analyses were then conducted for fMRI RS functional connectivity. These effects of tACS were evaluated with simple contrasts (paired t tests of pre versus post sessions) in the Active group (P<0.05). To control for time-related confounds, double contrasts of pre versus post between Active and Sham groups (Active-ShamPost-Pre; P<0.05) were also performed. Given the multiple ROIs considered in the analyses, FDR correction was applied to the double contrasts across the ROIs. Seed-based whole-brain connectivity was corrected with small-volume correction (FDR P<0.05) in SPM12. DMN source-level alpha-frequency GC was also submitted to the same simple and double contrasts. Finally, to link EEG and fMRI effects of tACS, the DMN source-level alpha-frequency GC was submitted into Pearson correlation analyses (P<0.05). Significant EEG-fMRI correlations were followed by mediation analyses to elucidate the contribution of alpha enhancement to α-tACS effects on DMN connectivity. The PROCESS macro for the Statistical Package for the Social Sciences (SPSS) was used to estimate 5,000 bias-corrected bootstrap samples, from which a 95% CI was created to test the indirect effect of α-tACS on DMN connectivity through alpha activity.

Targeted α-tACS Increases Alpha Activity and Connectivity

High-definition (HD) tACS was applied using a 4×1 montage over midline occipitoparietal sites, with 4 surrounding and 1 central electrodes forming a closed circuit (FIG. 1). These electrodes were selected to maximally target the primary alpha cortical source—the occipitoparietal cortex. Finite-element model simulation of the current distribution based on a standard head model confirmed maximal electric fields (0.21 V/m) in the occipitoparietal cortex (peaking at the occipito-parietal junction; x, y, z=16, −86, 36), relative to minimal electric fields (<0.02 V/m) in frontal regions. Another finite-element model estimation based on a realistic head model (the average T1 of the Active group) yielded a similar distribution of the current (FIG. 2). Source-level analysis of EEG alpha power in the Active group showed maximal alpha power increase in the right occipitoparietal cortex (FIG. 3A). In sum, these results confirmed that our tACS accurately targeted the occipitoparietal cortex.

Importantly, in support of the efficacy of α-tACS, increases in right posterior alpha power and right posterior-to-frontal (P→F) alpha connectivity was observed in the Active (versus Sham) group from pre- to post-stimulation (FIGS. 3B-3F). These effects, including the right-hemisphere dominance, replicated previous tACS findings. Specifically, after tACS, the Active group (n=17) showed significant increase in right posterior alpha power from the baseline (t=2.69, P=0.015), while no change was observed in the Sham group (n=19; P=0.92) (FIGS. 3B-3D). A double contrast (Post-PreActive-Sham) further confirmed specific alpha power increase in the Active (versus Sham) group (t=2.14, P=0.040). Left posterior alpha power also increased in the Active group (t=2.16, P=0.045), which, however, failed to survive the double contrast (t=1.36, P=0.184).

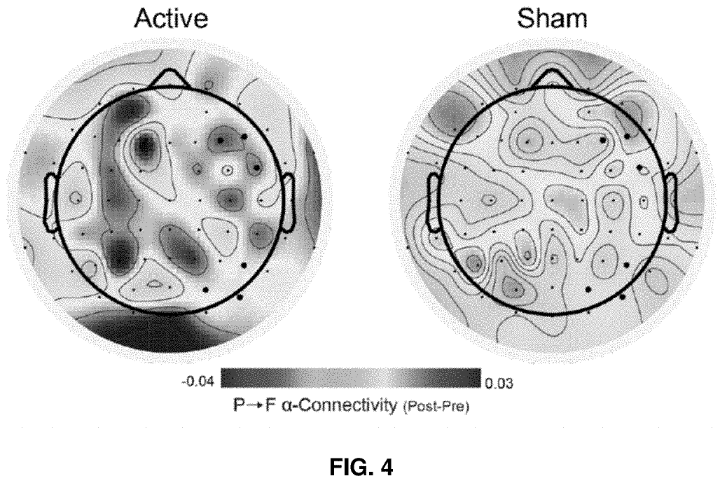

As evinced by recent neural computational and electrophysiological (including intracranial recordings) studies, alpha projections track a selective P→F direction (i.e., directed alpha P→F connectivity) via P→F cortical synchronization or traveling waves. Granger causality (GC) analysis was therefore to examine changes in this alpha connectivity. It was observed that the Active group also exhibited an increase in right-hemispheric alpha P→F connectivity (t=2.36, P=0.031), which was again absent in the Sham group (P=0.621) (FIGS. 3E-3F; FIG. 4). A similar double contrast confirmed that this alpha connectivity increase was specific to the Active group (t=2.12, P=0.042). Exploratory analyses of opposite F→P connectivity showed no effect of tACS. Finally, as illustrated in FIG. 3C and FIG. 3E, both power and connectivity increases were constrained to the alpha frequency, highlighting the specific tACS effects on alpha oscillations.

Targeted α-tACS Increases DMN Activity and Connectivity

### Increased DMN BOLD Connectivity

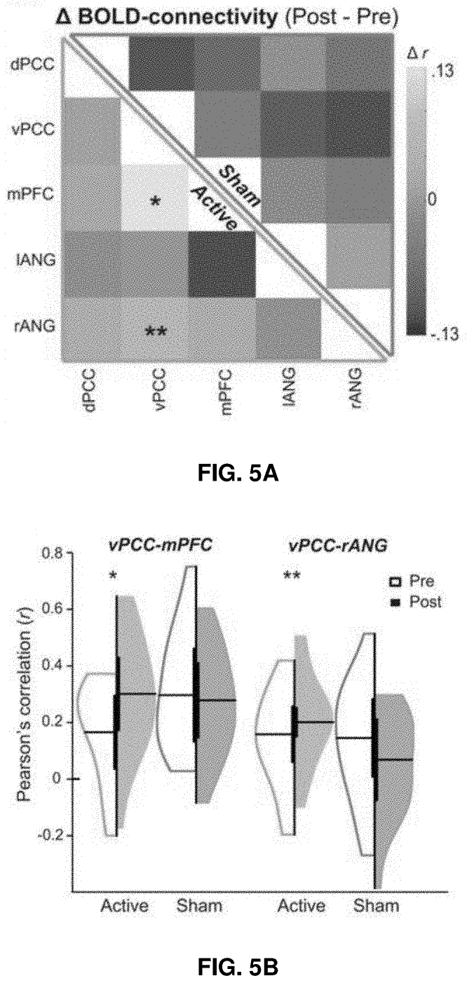

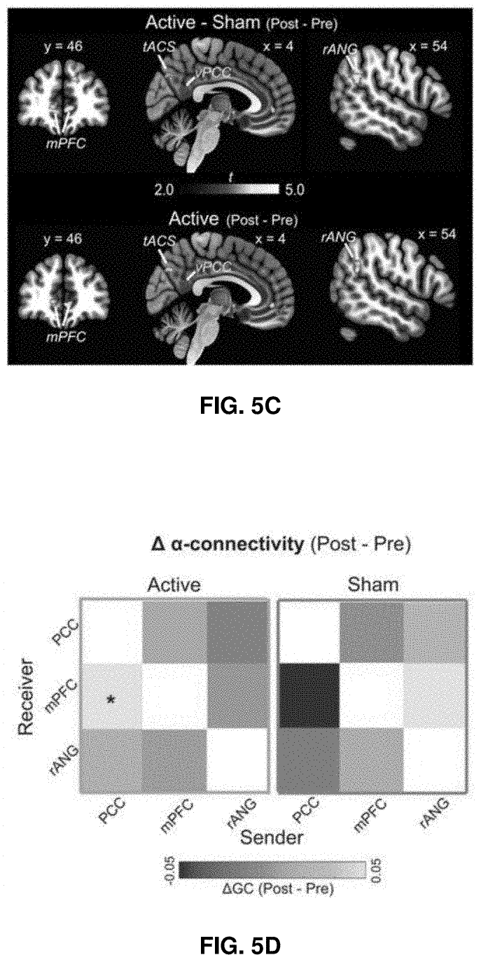

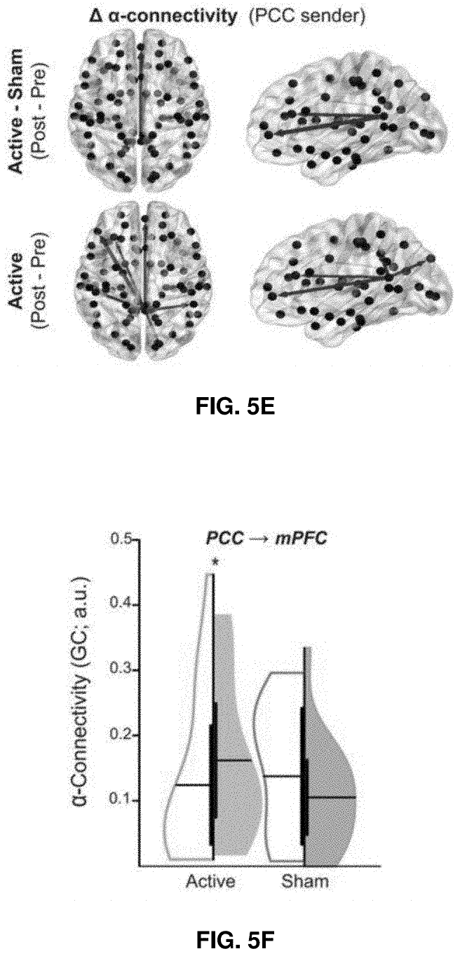

Extracting RS fMRI BOLD timeseries from the DMN regions of interest (ROIs)—midline hubs (mPFC and the ventral and dorsal subdivisions of PCC—vPCC and dPCC) and a key lateral node (left/right angular gyrus (ANG))—before and after stimulation, ROI-based functional connectivity analysis was conducted (followed by multiple comparison correction based on the false discovery rate (FDR)). The Active group demonstrated increases in vPCC-mPFC (t=2.87, P=0.011) and vPCC-rANG (t=3.12, P=0.007) connectivity after tACS (FIGS. 5A-5C). There was no change in the Sham group (p's>0.13), while double contrasts (Post-PreActive-Sham) further confirmed these increases were specific to the Active group (t's>2.79, p's<0.009, FDR P<0.05). Whole-brain vPCC-seed connectivity maps (of both simple and double contrasts) confirmed these results and, importantly, indicated that the increases were largely constrained to the DMN (FIG. 5C; FIG. 6)

### Increased DMN Alpha-Frequency Connectivity

The effect of tACS on source-level (DMN) alpha P→F connectivity was also examined A double contrast (Post-PreActive-Sham) showed a specific increase in alpha PCC→mPFC connectivity in the Active (versus Sham) group (t=2.27, P=0.030) (FIGS. 5D-5F) after stimulation. Specifically, the Active group showed an increase (t=1.74, P=0.049, one-tailed), while the Sham group trended toward a decrease (t=−1.48, P=0.079, one-tailed) in this connectivity. The alpha rANG→mPFC connectivity was not affected by tACS (P=0.92). Exploratory analyses of alpha horizontal connectivity between rANG and PCC (in both directions) and opposite F→P alpha connectivity (i.e., mPFC→PCC and mPFC→rANG) showed no effect of tACS (p's>0.440) Finally, in keeping with these ROI-based results, whole-brain maps of causal connectivity (GC) from the PCC indicated specific increase (in the Active versus Sham group) from the PCC to the mPFC, including the mPFC ROI and the rostral anterior cingulate cortex/ACC (FIG. 5E).

### Correlating Alpha Connectivity and DMN Connectivity

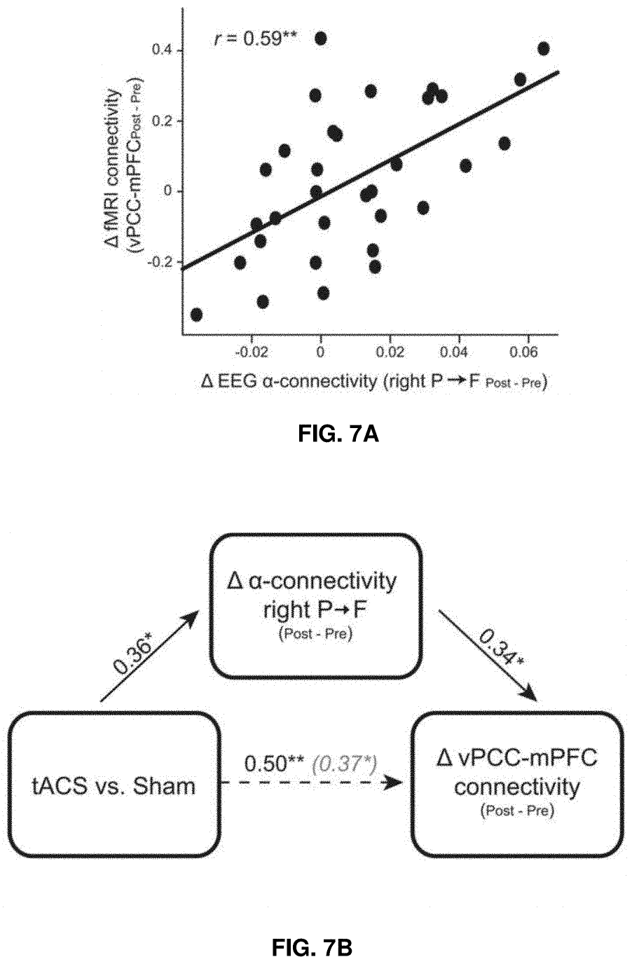

Correlational analysis showed that increases in BOLD vPCC-mPFC connectivity from the baseline strongly correlated with increases in alpha P→F connectivity (r=0.59, P<0.001) (FIG. 7A). Given the significant enhancement of right-hemisphere GC by tACS, this right lateral GC change was correlated with DMN connectivity change. Furthermore, this correlation was significant within the two groups individually (Active: r=0.51, P=0.030; Sham: r=0.49, P=0.047), highlighting the robust, inherent coupling between DMN and alpha connectivity. Changes in P→F connectivity in other (delta, theta, and beta) frequencies showed no such association (p's>0.111), demonstrating a unique association between changes in alpha-frequency connectivity and DMN connectivity. Furthermore, increases in BOLD vPCC-rANG did not correlate with alpha connectivity (r=0.14, P=0.545), highlighting the association of alpha P→F connectivity with DMN posterior—anterior connectivity. Importantly, a mediation analysis of tACS modulation of BOLD vPCC-mPFC connectivity revealed a significant indirect effect of alpha P→F connectivity (beta=0.076, CI=[0.018 0.189]), suggesting that increases in alpha P→F connectivity mediated the effect of tACS on BOLD vPCC-mPFC connectivity (FIG. 7B).

Results of Targeted α-tACS Isolated to the DMN

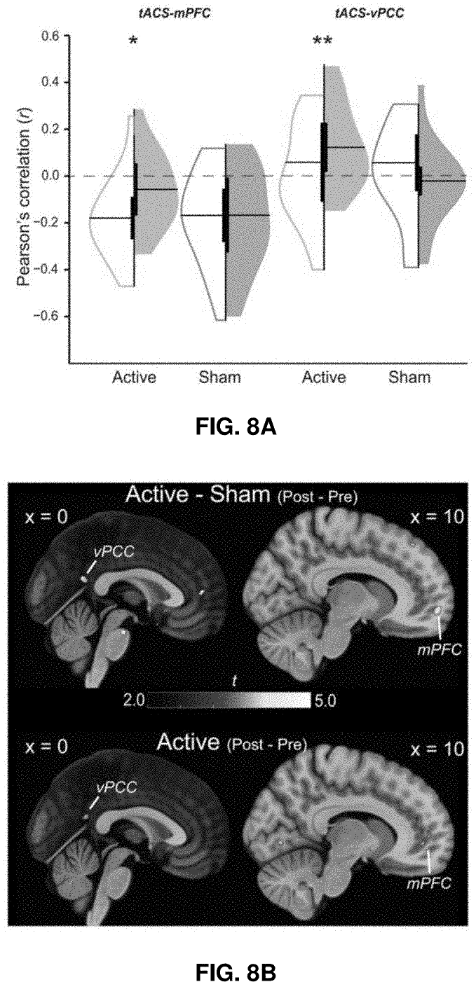

To attain mechanistic insights into tACS-induced neuromodulation, BOLD connectivity between the tACS site (a 10-mm sphere around the voxel with maximal electrical field of tACS) (FIG. 1) and the DMN was examined Active group demonstrated vPCC—tACSsite (site of tACS) connectivity increase after tACS (t=3.73, P=0.002), while the Sham group showed no change (P=0.578) (FIG. 8A). A double contrast further confirmed the specific increase in the Active (versus Sham) group (t=2.30, P=0.028). The Active group showed tACSsite-mPFC connectivity increase after tACS (t=2.89, P=0.011), while the Sham group showed no change (P=0.836). Again, a double contrast confirmed that the increase was specific to the Active (versus Sham) group (t=1.75, P=0.045 one-tailed). No effects of tACS emerged in the tACSsite-rANG connectivity (p's>0.272). Whole-brain tACSsite seed-based connectivity analysis confirmed these results while showing little connectivity change outside the DMN (FIG. 8B).

### Example 2. Replication and Control Study (HD EEG-tACS)

To replicate the findings and further specify α-tACS effects, an independent α-tACS study was conducted with an active control condition. In this study, participants (n=34) received the same α-tACS protocol as the main study (Example 1) and an active control condition—transcranial random noise stimulation (tRNS)—which was identical to the α-tACS condition except that the stimulation frequency varied randomly between 1 and 200 Hz. The experiment consisted of a 2-minute baseline eyes-open resting state, followed by 20 minutes of either α-tACS or tRNS and another (post-stimulation) resting state. After 10 minutes, participants then receive the alterative stimulation condition, followed by a final post-stimulation resting state. The order of stimulation (α-tACS/tRNS) condition was counterbalanced across participants. During the resting-state periods, high-density (hd) EEG recordings were acquired using a 96-channel (BrainProducts) system at a 2000 Hz sampling rate. The same EEG preprocessing (except cardioballistic artifact correction that was irrelevant here) and analysis as in the main study were then performed. Significant increases were found in right posterior alpha power (t=3.72, P=0.00073) and right-hemisphere P→F alpha connectivity (t=2.09, P=0.044) following a-tACS but not tRNS (p's>0.217). Similarly, at the source level, significant increase in alpha-frequency DMN (PCC→mPFC) connectivity were observed (t=2.28, P=0.029) after α-tACS but not tRNS (P=0.246).

### EMBODIMENTS

Some embodiments of the present disclosure can be described in view of one or more of the following:

Embodiment 1. A method of treating disorders associated with default mode network (DMN) dysfunction in a patient in need thereof, the method comprising administering alpha-frequency transcranial alternating current stimulation (α-tACS) to the brain of the patient via at least one electrode, wherein the α-tACS is effective to regulate DMN dysfunction and treat disorders associated therewith.

Embodiment 2. The method of Embodiment 1, wherein the associated with DMN dysfunction is selected from normal or abnormal aging, cognitive impairment, or neuropsychiatric disorders such as Alzheimer's disease, schizophrenia, autism, or posttraumatic stress disorder (PTSD).

Embodiment 3. The method of either of Embodiments 1 or 2, wherein the α-tACS is administered to the occipitoparietal cortex of the brain of the patient.

Embodiment 4. The method of any one of Embodiments 1 to 3, wherein the at least one electrode comprises a plurality of electrodes positioned on and/or around the midline of the occipitoparietal cortex.

Embodiment 5. The method of Embodiment 4, wherein the plurality of electrodes comprises a central electrode positioned on the midline of the occipitoparietal cortex, at least two right electrodes positioned right of the midline of the occipitoparietal cortex, and at least two left electrodes positioned left of the midline of the occipitoparietal cortex.

Embodiment 6. The method of either of Embodiments 4 or 5, wherein the plurality of electrodes form a closed-loop circuit.

Embodiment 7. The method of any one of Embodiments 1 to 6, wherein the a-tACS is administered to the patient's brain for at least 10 minutes, at least 20 minutes, or from 10 minutes to 20 minutes.

Embodiment 8. The method of any one of Embodiments 1 to 7, wherein the a-tACS comprises a sinusoidal current up to 3 mA, preferably 2 mA, oscillating at 8 Hz to 12 Hz, preferably 10 Hz.

Embodiment 9. A method of regulating default mode network (DMN) functioning in the brain of a patient, the method comprising forming a closed-loop circuit comprising two or more electrodes, and applying alpha-frequency transcranial alternating current stimulation (α-tACS) to the brain via the closed-loop circuit of electrodes, wherein the a-tACS targets a primary rhythm of neural synchrony in the brain associated with DMN functioning.

Embodiment 10. The method of Embodiment 9, wherein the closed-loop circuit of electrodes comprises a central electrode and four peripheral electrodes.

Embodiment 11. The method of either of Embodiments 9 or 10, wherein the primary rhythm of neural synchrony is the alpha-frequency, preferably 8 to 12 Hz oscillations in the brain.

Embodiment 12. The method of any one of Embodiments 9 to 11, wherein the a-tACS comprises a 2 mA sinusoidal current oscillating at 10 Hz.

Embodiment 13. The method of any one of Embodiments 9 to 12, wherein the a-tACS is applied via the closed-loop circuit of electrodes for at least 10 minutes, preferably 20 minutes.

Embodiment 14. The method of any one of Embodiments 9 to 13, wherein regulating DMN functioning is effective to treat normal or abnormal aging, cognitive impairment, or neuropsychiatric disorders such as Alzheimer's disease, schizophrenia, autism, or posttraumatic stress disorder (PTSD).

Embodiment 15. A system for delivering alpha-frequency transcranial alternating current stimulation (α-tACS) to the brain of a patient in need thereof, the system comprising at least one electrode configured to be removably attachable to the patient, and an apparatus configured to be operably connected to the at least one electrode and to generate α-tACS and deliver the α-tACS, via the at least one electrode, to the patient's brain, wherein the system is configured to target α-tACS a primary rhythm of neural synchrony in the brain associated with DMN functioning.

Embodiment 16. The system of Embodiment 15, wherein the at least one electrode is a plurality of electrodes configured to target the occipitoparietal cortex of the brain.

Embodiment 17. The system of either of Embodiments 15 or 16, wherein the plurality of electrodes comprises a central electrode configured to target the midline of the occipitoparietal cortex, at least one right electrode configured to target a portion of the occipitoparietal cortex to the right of the midline, and at least one left electrode configured to target a portion of the occipitoparietal cortex to the left of the midline.

Embodiment 18. The system of Embodiment 17, wherein the central electrode is configured to deliver a sinusoidal current up to 3 mA, preferably 2 mA, and wherein the at least one right electrode and the at least one left electrode are configured to deliver a net sinusoidal current up to 1 mA, preferably 0.5 mA.

Embodiment 19. The system of any one of Embodiments 15 to 18, wherein the at least one electrode is configured to deliver a 2 mA sinusoidal current oscillating at 10 Hz.

That which is claimed is:

1. A method of treating disorders associated with default mode network (DMN) dysfunction in a patient in need thereof, the method comprising:
administering alpha-frequency transcranial alternating current stimulation (α-tACS) to the brain of the patient via at least one electrode,
wherein the α-tACS is effective to regulate DMN dysfunction and treat disorders associated therewith.

2. The method of claim 1, wherein the disorder associated with DMN dysfunction is selected from normal or abnormal aging, cognitive impairment, or neuropsychiatric disorders such as Alzheimer's disease, schizophrenia, autism, or posttraumatic stress disorder (PTSD).

3. The method of claim 1, wherein the α-tACS is administered to the occipitoparietal cortex of the brain of the patient.

4. The method of claim 3, wherein the at least one electrode comprises a plurality of electrodes positioned on and/or around the midline of the occipitoparietal cortex.

5. The method of claim 4, wherein the plurality of electrodes comprises:
a central electrode positioned on the midline of the occipitoparietal cortex;
at least two right electrodes positioned right of the midline of the occipitoparietal cortex; and
at least two left electrodes positioned left of the midline of the occipitoparietal cortex.

6. The method of claim 4, wherein the plurality of electrodes form a closed-loop circuit.

7. The method of claim 1, wherein the α-tACS is administered to the patient's brain for a period at least 10 minutes or least 20 minutes.

8. The method of claim 1, wherein the α-tACS comprises sinusoidal current up to 3 mA, preferably 2 mA, oscillating at 8 Hz to 12 Hz, preferably 10 Hz.

9. A method of regulating default mode network (DMN) functioning in the brain of a patient, the method comprising:
forming a closed-loop circuit comprising two or more electrodes; and
applying alpha-frequency transcranial alternating current stimulation (a-tACS) to the brain via the closed-loop circuit of electrodes,
wherein the α-tACS targets a primary rhythm of neural synchrony in the brain associated with DMN functioning.

10. The method of claim 9, wherein the closed-loop circuit of electrodes comprises a central electrode and four peripheral electrodes.

11. The method of claim 9, wherein the primary rhythm of neural synchrony is the alpha-frequency, preferably 8 to 12 Hz oscillations in the brain.

12. The method of claim 9, wherein the α-tACS comprises a 2 mA sinusoidal current oscillating at 10 Hz.

13. The method of claim 9, wherein the α-tACS is applied via the closed-loop circuit of electrodes for at least 10 minutes or at least 20 minutes.

14. The method of claim 9, wherein regulating DMN functioning is effective to treat normal or abnormal aging, cognitive impairment, or a neuropsychiatric disorder selected from Alzheimer's disease, schizophrenia, autism, and post-traumatic stress disorder (PTSD).

15. A system for delivering alpha-frequency transcranial alternating current stimulation (α-tACS) to the brain of a patient in need thereof, the system comprising:
at least one electrode configured to be removably attachable to the patient; and
an apparatus configured to be operably connected to the at least one electrode and to generate α-tACS and deliver the α-tACS, via the at least one electrode, to the patient's brain,
wherein the system is configured to target α-tACS a primary rhythm of neural synchrony in the brain associated with DMN functioning.

16. The system of claim 15, wherein the at least one electrode is a plurality of electrodes configured to target the occipitoparietal cortex of the brain.

17. The system of claim 15, wherein the plurality of electrodes comprises:
a central electrode configured to target the midline of the occipitoparietal cortex;
at least one right electrode configured to target a portion of the occipitoparietal cortex to the right of the midline; and
at least one left electrode configured to target a portion of the occipitoparietal cortex to the left of the midline.

18. The system of claim 17, wherein the central electrode is configured to deliver a sinusoidal current up to 3 mA, preferably 2 mA, and wherein the at least one right electrode and the at least one left electrode are configured to deliver a net sinusoidal current up to 1 mA, preferably 0.5 mA.

19. The system of claim 15, wherein the at least one electrode is configured to deliver a 2 mA sinusoidal current oscillating at 10 Hz.
